(19) United States
(12) Patent Application Publication
(10) Pub. No.: US 2019/0224137 A1
(43) Pub. Date: Jul. 25, 2019

(54) Cannabinoid Dosing Regime for Acne

(71) Applicant: Botanix Pharmaceuticals Ltd., Plymouth Meeting, PA (US)

(72) Inventor: Matthew Callahan, Haverford, PA (US)

(73) Assignee: Botanix Pharmaceuticals Ltd., Plymouth Meeting, PA (US)

(21) Appl. No.: 16/257,031

(22) Filed: Jan. 24, 2019

Related U.S. Application Data
(60) Provisional application No. 62/621,274, filed on Jan. 24, 2018.
(60) Provisional application No. 62/671,542, filed on May 15, 2018.
(60) Provisional application No. 62/736,024, filed on Sep. 25, 2018.

(51) Int. Cl. A61K 31/047; A61P 17/10; A61K 9/06; A61K 9/00; A61K 47/24; A61K 47/10

(52) U.S. Cl. CPC A61K 31/047; A61P 17/10; A61K 47/10; A61K 9/0014; A61K 47/24; A61K 9/06

(57) ABSTRACT

A method for treat acne comprising administering between 50 mg and 3000 mg of a topical liquid or gel composition comprising between 1% w/w and 15% w/w cannabinoid, wherein the cannabinoid is dissolved in the liquid or gel composition is described.

### RELATED APPLICATIONS

This application claims priority under 35 USC § 119(e) to U.S. Patent Application Ser. No. 62/621,274, filed on Jan. 24, 2018, U.S. Patent Application Ser. No. 62/671,542, filed on May 15, 2018, and U.S. Patent Application Ser. No. 62/736,024, filed on Sep. 25, 2018, the entire contents of which are hereby incorporated by reference.

### TECHNICAL FIELD

A topical dosing regimen for the treatment or prevention of acne using cannabinoids.

### BACKGROUND ART

Most mammalian skin, including human skin, comprises three layers: (i) an epidermis layer; (ii) a dermis layer; and (iii) a hypodermis layer. The epidermis itself is made up of two layers, the outer stratum corneum and the inner epidermal basal layer.

Acne is a multi-factorial disease affecting the sebaceous follicle and characterized by papules, pustules, and scars. Acne affects more than 80% of 16-year old boys and girls, but is not a problem confined to teenagers. Simple attention to hygiene is no longer sufficient and antiseptic washes, so popular some years ago, are now perceived as ineffective by many sufferers and most clinicians.

Effective management of acne can be accomplished by addressing the four key features of the pathogenesis. Topical therapy is usually the first choice for patients. The use of topical therapy minimizes potential side effects associated with the use of systemic agents.

Because acne is a multi-factorial disease which is manifest to varying degrees, it is important for the physician to assess the patient to attempt to find therapies which will be helpful to the patient without causing major side effects. All of the current conventional treatments are associated with some degree of adverse side effects that limit their usefulness.

Cannabinoids have been proposed as a treatment for skin conditions such as acne. However, the amount of active agent in the available topical creams is usually very low, and there is little evidence that a therapeutically useful dose is being provided to the user.

It is against this background that the present invention has been developed. The present invention seeks to provide a high dosage composition of cannabinoids for topical use to treat or prevent acne, or to provide the consumer with a useful therapeutic or commercial choice.

The previous discussion of the background art is intended to facilitate an understanding of the present invention only. The discussion is not an acknowledgement or admission that any of the material referred to is, or was part of the common general knowledge, as at the priority date of the application.

### SUMMARY OF INVENTION

In accordance with the present invention, there is provided a regime for use in the treatment or prevention of acne, said regime comprising the administration of:
a) between 50 mg and 3000 mg of a topical composition comprising between 1% w/w and 15% w/w cannabinoid to the skin of a subject in need of such treatment or prevention.

Preferably, the composition comprises between 2% w/w and 7% w/w cannabinoid, more preferably 2.5% w/w or 5% w/w.

Preferably, the composition of the treatment regime is administered to the skin between 1 and 5 times per day, more preferably once or twice per day.

Preferably, the composition of the treatment regime delivers between 20 mg and 400 mg of cannabinoid per administration, more preferably, 37.5 mg or 75 mg of cannabinoid per administration.

Preferably, the total daily dose applied to the skin is between 20 mg and 2000 mg cannabinoid, more preferably 37.5 mg, 75 mg, or 150 mg.

Preferably, the composition of the treatment regime is in a liquid or gel form.

The present invention further provides a method for treating or preventing acne, said method comprising the administration of:
a) between 50 mg and 3000 mg of a topical composition comprising between 1% w/w and 15% w/w cannabinoid to the skin of a subject in need of such treatment or prevention.

The present invention further provides for the use of between 50 mg and 3000 mg of a topical composition comprising between 1% w/w and 15% w/w cannabinoid for the treatment or prevention of acne in a subject in need of such treatment or prevention.

The present invention further provides for the use of between 1% w/w and 15% w/w cannabinoid for the manufacture of a topical composition for the treatment or prevention of acne, wherein between 50 mg and 3000 mg of the topical composition is administered to the skin of a subject in need of such treatment or prevention.

The present invention further provides for the manufacture of a topical composition comprising between 1% w/w and 15% w/w cannabinoid for use in the treatment or prevention of acne, wherein between 50 mg and 3000 mg of the topical composition is administered to the skin of a subject in need of such treatment or prevention.

The present invention further provides a topical composition comprising between 1% w/w and 15% w/w cannabinoid for use in the treatment or prevention of acne, wherein between 50 mg and 3000 mg of the topical composition is administered to the skin of a subject in need of such treatment or prevention.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a graph of the percent changes in lesion counts resulting from an Open-Label Study to Evaluate the Safety and Tolerability of BTX 1503 Solution in Patients with Acne Vulgaris.

### DESCRIPTION OF INVENTION

### Detailed Description of the Invention

The present invention is based on the finding that the amount of cannabinoids in the available topical creams for acne treatment is usually very low, and there is little evidence that a therapeutically useful dose is being provided to the user. The average topical cannabinoid cream is labelled to contain between about 300 mg and 750 mg of cannabinoid per 120 mL jar of cream, which if the labelling is correct, provides an average dose, once applied to the skin, of about 5 mg to 15 mg per dose.

The term cannabinoid includes compounds which interact with the cannabinoid receptor and various cannabinoid mimetics, such as certain tetrahydropyran analogs (e.g., Δ⁹-tetrahydrocannabinol, Δ⁸-tetrahydro-cannabinol, 6,6,9-trimethyl-3-pentyl-6H-dibenzo[b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-Δ6-tetrahydrocannabinol-1,1-dimethylheptyl,(+)-(3S,4S)-7-hydroxy-Δ6-tetrahydrocannabinol-1,1-dimethylheptyl, 11-hydroxy-Δ⁹-tetrahydrocannabinol, and Δ8-tetrahydrocannabinol-11-oic acid)); certain piperidine analogs (e.g., (−)-(6S,6aR,9R,10aR)-5,6,6a,7,8,9,10,10a-octahydro-6-methyl-3-[(R)-1-methyl-4-phenylbutoxy]-1,9-phenanthridinediol-1-acetate)); certain aminoalkylindole analogs (e.g., (R)-(+)-[2,3-dihydro-5-methyl-3-(-4-morpholinylmethyl)-pyrrolo[1,2,3-de]-1,4-benzoxazin-6-]-1-naphthalenyl-methanone), and certain open pyran ring analogs (e.g., 2-[3-methyl-6-(1-methylethenyl)-2-cyclohexen-1-yl]-5-pentyl-1,3-benzenediol and 4-(1,1 -dimethylheptyl)-2,3′-dihydroxy-6′alpha-(3-hydroxypropyl)-1′,2′,3′,4′,5′,6′-hexahydrobiphenyl).

Cannabidiol (CBD), as used herein, refers to 2-[3-methyl-6-(1-methylethenyl)-2-cyclohexen-1-yl]-5-pentyl-1,3-benzenediol. The synthesis of cannabidiol is described, for example, in Petilka et al., Helv. Chim.Acta, 52: 1102 (1969) and in Mechoulam et al., J. Am. Chem. Soc., 87:3273 (1965), which are hereby incorporated by reference

Identification of the main cannabinoid receptors (CB1 and CB2), their endogenous lipid ligands (endocannabinoids), biosynthetic pathways and metabolizing enzymes (collectively termed the ECS), coupled with the discovery and/or rational design of numerous exogenous ligands for CB receptors, has triggered an exponential growth in studies exploring the continuously growing regulatory functions of this newly discovered physiological system both in health and disease.

The most extensively studied endocannabinoids are anandamide (N arachidonoylethanolamine, AEA) and 2-arachidonoylglycerol (2-AG). Multiple pathways are involved in synthesis and cellular uptake of these lipid mediators. The most common degradation pathways for AEA and 2-AG are the fatty acid amid hydrolase (FAAH) and monoacylglycerol lipase (MAGL) enzyme. Endocannabinoids, similar to Δ⁹-tetrahydrocannabinol (THC; the main active ingredient of the plant Cannabis sativa), predominantly exert their physiological effects via two main G-protein-coupled cannabinoid receptors; however, numerous additional signalling mechanisms and receptor systems (e.g. transient receptor potential cation channel, subfamily V, member 1; TRPV1) might also be involved. Initially, the CB1-mediated effects were described centrally and CB1 receptors were thought to be restricted to the central nervous system, whereas CB2 was first identified at the periphery in immune cells.

It is considered that CBD may:
normalise excessive lipid synthesis of human sebocytes (the cells from the oil producing sebaceous glands in the skin which disintegrate and release their oil content);
decrease proliferation (but not the viability) of these human sebocytes;
inhibit hyperproliferation of keratinocytes; and
exert universal anti-inflammatory actions.

Without being held to any theory, we believe that the mode of action of CBD for anti-acne activity involves the suppression of mediators of inflammatory responses. CBD has been shown to have lipostatic, anti-proliferative, and anti-inflammatory effects on immortalized human sebocytes. There is a physiological regulatory function of the endocannabinoid system (ECS) in proliferation, differentiation, apoptosis and cytokine, mediator and hormone production of various cell types of the skin and appendages (e.g. hair follicle, sebaceous gland), and there is evidence on the putative involvement of the ECS in certain pathological conditions of the skin including acne and seborrhea [Biro, 2009].

In vitro studies have shown CBD to stimulate the human vanilloid receptor type 1 (VR1) and to inhibit anandamide (an endogenous CBD neurotransmitter). These findings have suggested a mode of action for the anti-inflammatory properties of CBD.

In vivo studies with intravenous administration of CBD in sensitized guinea-pigs reduced airway obstruction, indicating a potential role of CBD in reducing immune-induced inflammatory reactions. Similarly, CBD injected into rats attenuated cardiac inflammation.

Treatment Regime

In contrast to the prior art, the present invention provide a regime for use in the treatment or prevention of acne, said regime comprising the administration of:
a) between 50 mg and 3000 mg of a topical composition comprising between 1% w/w and 15% w/w cannabinoid to the skin of a subject in need of such treatment or prevention.

Preferably the topical composition comprising between 1% w/w and 15% w/w cannabinoid is a liquid or gel composition.

Preferably, an amount of between 50 mg and 3000 mg, between 50 mg and 2000 mg, between 50 mg and 1000 mg, between 50 mg and 500 mg, between 50 mg and 400 mg, between 50 mg and 300 mg, between 50 mg and 200 mg, between 50 mg and 100 mg of the composition may be administered to the skin of the subject in each administration. For example, 50 mg, 100 mg, 200 mg, 300 mg, 400 mg, 500 mg, 600 mg, 700 mg, 800 mg, 900 mg, 1000 mg, 1500 mg, 2000 mg, 2500 mg or 3000 mg of the composition may be administered to the skin of the subject in each administration. Preferably an amount of about 100 mg is administered to the skin of the subject in each administration.

Preferably, an amount of between 50 mg and 3000 mg, between 50 mg and 2000 mg, between 50 mg and 1000 mg, between 50 mg and 500 mg, between 50 mg and 400 mg, between 50 mg and 300 mg, between 50 mg and 200 mg, between 50 mg and 100 mg of the composition may be administered to the face of the subject in each administration. For example, 50 mg, 100 mg, 200 mg, 300 mg, 400 mg, 500 mg, 600 mg, 700 mg, 800 mg, 900 mg, 1000 mg, 1500 mg, 2000 mg, 2500 mg or 3000 mg of the composition may be administered to the face of the subject in each administration. Preferably an amount of about 100 mg is administered to the face of the subject in each administration.

Preferably, an amount of between 50 mg and 3000 mg, between 50 mg and 2000 mg, between 50 mg and 1000 mg, between 50 mg and 500 mg, between 50 mg and 400 mg, between 50 mg and 300 mg, between 50 mg and 200 mg, between 50 mg and 100 mg of the composition may be administered to 565 cm² of skin of the subject in each administration. For example, 50 mg, 100 mg, 200 mg, 300 mg, 400 mg, 500 mg, 600 mg, 700 mg, 800 mg, 900 mg, 1000 mg, 1500 mg, 2000 mg, 2500 mg or 3000 mg of the composition may be administered to 565 cm² of skin of the subject in each administration. Preferably an amount of about 100 mg is administered to 565 cm² of the subject in each administration.

Preferably the composition comprises between 1% w/w and 15% w/w cannabinoid, between 1% w/w and 14% w/w, between 1% w/w and 13% w/w, between 1% w/w and 12% w/w, between 1% w/w and 11% w/w, between 1% w/w and 10% w/w, between 1% w/w and 9% w/w, between 1% w/w and 8% w/w, between 1% w/w and 7% w/w, between 1% w/w and 6% w/w, between 1% w/w and 5% w/w, between 2% w/w and 5% w/w, between 2% w/w and 4% w/w, between 3% w/w and 5% w/w, between 4% w/w and 5% w/w cannabinoid. For example, the composition may comprise 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, or 15% w/w cannabinoid

In certain embodiments, the concentration of cannabinoid in the topical composition of the invention may be selected from the group consisting of: at least 2% w/w, at least 3% w/w, at least 4% w/w, at least 5% w/w, at least 6% w/w, at least 7% w/w, at least 8% w/w, at least 9% w/w, at least 10% w/w, at least 11% w/w, at least 12% w/w, at least 13% w/w, at least 14% w/w, and at least 15% w/w.

In certain embodiments, the concentration of cannabinoid in the topical composition may be within a range with a lower limit selected from the group consisting of: 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, and 15% w/w; and an upper limit selected from the group consisting of: 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w and 15% w/w.

More preferably, the concentration of cannabinoid in the topical composition is 2.5% w/w or 5% w/w.

Preferably, the composition of the treatment regime delivers between 20 mg and 400 mg of cannabinoid per administration. For example, the composition of the treatment regime deliver may between 20 mg and 400 mg, 20 mg and 350 mg, 20 mg and 300 mg, 20 mg and 250 mg, 20 mg and 200 mg, 20 mg and 150 mg, 20 mg and 100 mg, 20 mg and 50 mg, 30 mg and 100 mg, 40 mg and 100 mg, 50 mg and 100 mg, 60 mg and 100 mg, 70 mg and 100 mg, 80 mg and 100 mg of cannabinoid per administration.

In certain embodiments, the composition of the treatment regime delivers an amount of cannabinoid per administration with a lower limit selected from the group consisting of: 20 mg, 30 mg, 40 mg, 50 mg, 60 mg, 70 mg, 80 mg, 90 mg, 100 mg, 110 mg, 120 mg, 130 mg, 140 mg, 150 mg, 200 mg, 250 mg, 300 mg and 350 mg; and an upper limit selected from the group consisting of: 30 mg, 40 mg, 50 mg, 60 mg, 70 mg, 80 mg, 90 mg, 100 mg, 110 mg, 120 mg, 130 mg, 140 mg, 150 mg, 200 mg, 250 mg, 300 mg, 350 mg and 400 mg.

More preferably, the amount of cannabinoid per administration is 37.5 mg or 75 mg.

In accordance with certain embodiments, the composition is applied to the affected area regularly until relief is obtained. In one preferred embodiment, the composition is administered to the skin of the patient in need of such treatment using a dosing regimen selected from the group consisting of: every hour, every 2 hours, every 3 hours, once daily, twice daily, three times daily, four times daily, five times daily, once weekly, twice weekly, once fortnightly and once monthly. However, other application schedules may be utilized in accordance with the present invention. Preferably, the composition of the treatment regime is administered to the skin between 1 and 5 times per day, more preferably once or twice per day.

Preferably the total daily dose applied to the skin by administration of the topical composition is between 20 mg and 2000 mg cannabinoid, preferably 20 mg and 2000 mg, 50 mg and 1500 mg, 20 mg and 200 mg, 100 mg and 1000 mg, 150 mg and 500 mg, 200 mg and 500 mg, 200 mg and 400 mg of cannabinoid.

In certain embodiments, the total daily dose of cannabinoid applied to the skin by administration of the topical composition has a lower limit selected from the group consisting of: 20 mg, 30 mg, 35 mg, 40 mg, 45 mg, 50 mg, 60 mg, 70 mg, 80 mg, 90 mg, 100 mg, 110 mg, 120 mg, 130 mg, 140 mg, 150 mg, 160 mg, 170 mg, 200 mg, 210 mg, 220 mg, 230 mg, 240 mg, 250 mg, 260 mg, 270 mg, 280 mg, 290 mg, 300 mg, 320 mg, 350 mg, 400 mg, 500 mg, 600 mg, 700 mg, 800 mg, 900 mg, 1000 mg, 1500 mg and 1900 mg; and an upper limit selected from the group consisting of: 30 mg, 50 mg, 70 mg, 100 mg, 150 mg, 200 mg, 210 mg, 220 mg, 230 mg, 240 mg, 250 mg, 260 mg, 270 mg, 280 mg, 290 mg, 300 mg, 320 mg, 350 mg, 400 mg, 500 mg, 600 mg, 700 mg, 800 mg, 900 mg, 1000 mg, 1500 mg and 2000 mg.

Most preferably, the total daily dose of cannabinoid applied to the skin by administration of the topical composition is 37.5 mg, 75 mg, or 150 mg.

Thus in relation to the compositions of the present invention, preferably:
an amount of between 50 mg and 3000 mg of the composition is administered to the skin;
the administered composition contains between 1% and 15% cannabinoid;
the administered composition delivers between 20 mg and 400 mg cannabinoid;
the composition is administered between 1 and 5 times per day; and
the total daily dose applied to the skin is between 20 mg and 2000 mg cannabinoid.

More preferably:
an amount of between 100 mg and 120 mg of the composition is administered to the skin;
the administered composition contains between 2.5% and 5% cannabinoid;
the administered composition delivers between 20 mg and 100 mg cannabinoid;
the composition is administered one or two times per day; and
the total daily dose applied to the skin is between 20 mg and 200 mg cannabinoid.

Most preferably:
an amount of between 100 mg and 120 mg of the composition is administered to the skin;
the administered composition contains 2.5% or 5% cannabinoid;
the administered composition delivers 37.5 mg or 75 mg cannabinoid;
the composition is administered one or two times per day; and
the total daily dose applied to the skin is between 37.5 mg and 150 mg cannabinoid.

High concentrations of cannabinoids delivered to the skin are expected to be advantageous in terms of enhancing the relevant extent of delivery into the skin, particularly the epidermis (including the epidermal basal layer), with some penetration into the dermis. It is thought that the high concentration of cannabinoids on the outer surface of the skin causes a concentration gradient that enhances penetration of the cannabinoid into the skin, particularly the epidermis and the dermis.

In order to achieve local distribution for the treatment of acne, it is advantageous for the majority of the cannabinoid, such as cannabidiol (CBD), to penetrate into the epidermis and preferably remain there, and for some cannabinoid to further penetrate to the dermis and the hypodermal layer to be absorbed systemically. In such a case, the cannabidiol would concentrate mainly in the epidermis, thus maximizing its local effect. Not only does the localized effect increase the potential therapeutic benefit, it potentially lessens the frequency and severity of any potential side-effects associated with systemic cannabinoid administration, because the amount of active compound circulating in the patient is reduced.

### Acne Treatment and Therapy

In certain embodiments the topical application of cannabinoid, such as cannabidiol, by way of the compositions of the present invention is expected to reduce the incidence and/or severity of acne. Therapeutic effects of the present invention include, but are not limited to, reduction in redness, itch, pain or irritation, a reduction in pimples, papules, blisters or pustules, a reduction in infection, a reduction of swelling, cracking, weeping, crusting, and scaling and/or a general decrease in inflammation.

In certain embodiments, the topical application of cannabinoid, such as cannabidiol, by way of the compositions of the present invention is expected to improve the symptoms of acne.

The term “improve” is used to convey that the present invention changes either the appearance, form, characteristics and/or the physical attributes of the tissue to which it is being provided, applied or administered. The change in form may be demonstrated by any of the following alone or in combination: enhanced appearance of the skin; decreased inflammation of the skin, prevention of inflammation or blisters, decreased spread of blisters, decreased ulceration of the skin, decreased redness, reduction of scarring, reduction in lesions, healing of blisters, reduced skin thickening, closure of wounds and lesions, a reduction in symptoms including, but not limited to, pain, inflammation, itching, milia or other symptoms associated with inflammatory conditions or the like.

A primary advantage of the present invention is expected to be the improvement in the condition of the skin without the typical side effects of conventional therapies. The potential for the present invention is widespread, and the topical application of cannabinoids shows promise as an exciting new method of acne treatment.

It is expected that treatment of acne in accordance with embodiments of the present invention results in improved healing of the skin. For example, when used in the treatment of acne, swollen, cracked or scaled skin is which is treated is expected to heal more quickly and/or completely, compared to when left untreated.

When administered in accordance with the present invention, treatment is expected to result in one or more therapeutic effects. Therapeutic effects in the affected area include, but are not limited to, reduction in redness, itch, pain or irritation, the number and severity of the acne lesions, a reduction in infection, a reduction of swelling, cracking, weeping, crusting, and scaling and/or a general decrease in inflammation. One or more of these therapeutic effects are expected to be observed when treatment in accordance with the present invention is made to any of the suitable conditions.

The present invention therefore provides a method for treating or preventing acne, said method comprising the administration of:
a) between 50 mg and 3000 mg of a topical composition comprising between 1% w/w and 15% w/w cannabinoid to the skin of a subject in need of such treatment or prevention.

Preferably the topical composition comprising between 1% w/w and 15% w/w cannabinoid is a liquid or gel composition. Preferably the composition is non-aqueous.

The present invention further provides for the use of between 50 mg and 3000 mg of a topical composition comprising between 1% w/w and 15% w/w cannabinoid for the treatment or prevention of acne in a subject in need of such treatment or prevention.

The present invention further provides for the use of between 1% w/w and 15% w/w cannabinoid for the manufacture of a topical composition for the treatment or prevention of acne, wherein between 50 mg and 3000 mg of the topical composition is administered to the skin of a subject in need of such treatment or prevention.

In one aspect, the present invention is directed to methods of treating acne using topical cannabinoids, including cannabidiol. In accordance with certain embodiments, a topical composition of the invention containing cannabinoids such as cannabidiol, is preferably applied topically to an area which is affected by acne. Preferably, the application of cannabinoid in accordance with certain embodiments results in reduction in redness, itch, pain or irritation, a reduction in pimples, papules, blisters or pustules, a reduction in infection, less breakdown and loss of collagen and elastin in the skin, a reduction of swelling, cracking, weeping, crusting, and scaling and/or a general decrease in inflammation.

Thus in relation to the methods of the present invention, preferably:
an amount of between 50 mg and 3000 mg of the composition is administered to the skin;
the administered composition contains between 1% and 15% cannabinoid;
the administered composition delivers between 20 mg and 400 mg cannabinoid;
the composition is administered between 1 and 5 times per day; and
the total daily dose applied to the skin is between 20 mg and 2000 mg cannabinoid.

More preferably:
an amount of about 100 mg of the composition is administered to the skin;
the administered composition contains between 2.5% and 5% cannabinoid;
the administered composition delivers between 20 mg and 100 mg cannabinoid;
the composition is administered one or two times per day; and
the total daily dose applied to the skin is between 20 mg and 200 mg cannabinoid.

Most preferably:
an amount of between 100 mg and 120 mg of the composition is administered to the skin;
the administered composition contains 2.5% or 5% cannabinoid;
the administered composition delivers 37.5 mg or 75 mg cannabinoid;
the composition is administered one or two times per day; and
the total daily dose applied to the skin is between 37.5 mg and 150 mg cannabinoid.

Pharmaceutical Compositions

The present invention provides a composition comprising between 1% w/w and 15% w/w cannabinoid for use in the treatment or prevention of acne, wherein between 50 mg and 3000 mg of the topical composition is administered to the skin of a subject in need of such treatment or prevention. Preferably the composition is administered to the skin between 1 and 5 times per day and preferably the total daily dose applied to the skin by administration of the topical composition is between 20 mg and 2000 mg cannabinoid.

Preferably there is a therapeutically effective amount of cannabinoid in each topical dose of the composition of the present invention. Therapeutically effective amount means the amount necessary to bring about a therapeutic effect.

Certain embodiments of the present invention comprise any topically acceptable carrier vehicle. Preferred topically acceptable vehicles include but are not limited to gels, ointments, and liquids. Administration of the preferred embodiment is performed in accordance with that mode which is most amenable to the topically acceptable form chosen. For example, gels, lotions, creams and ointments are preferably administered by spreading. The topical composition may or may not contain water, i.e. it may be an aqueous or a non-aqueous composition.

The dilution of the cannabinoid in the topical composition can be an important consideration. The cannabinoid concentration in the composition should be high enough that the patient does not need to wait an excessively long time for the composition to dry. On the other hand, the cannabinoid concentration should be dilute enough that a patient can achieve effective coverage of the affected area. Additionally, the composition could include a component which polymerizes in response to exposure to air or ultraviolet radiation.

The amount of composition to be applied will vary. When the cannabinoid, such as cannabidiol, is administered by spraying a solution of the drug, the total volume in a single dose may be as low as 0.1 ml. When the cannabinoid, such as cannabidiol, is administered in a gel or cream, the total volume may be as high as 3 ml. Conversely, if acne comprises scattered lesions, the volume applied to each lesion may be smaller. The carrier selected, and its manner of application, are preferably chosen in consideration of the needs of the patient and the preferences of the administering physician.

In one preferred embodiment, the composition comprises a gel which is preferably administered by spreading the gel onto the affected area. In other preferred embodiments, the composition comprises a liquid, which can be administered by spraying or otherwise applying the liquid onto the affected area.

In certain embodiments, the composition of the invention may be provided in a form selected from the group comprising, but not limited to a liquid, cream or gel. The composition may be a leave-on preparation, or a wash-off preparation. In one preferred form, the composition is a cream or gel. In another preferred form, the composition is a spray. The composition may or may not contain water. Preferably, the composition does not contain water, i.e. it is non-aqueous.

The cannabinoid could be incorporated into a composition with an additional active moiety that is capable of improving the appearance and/or hydration of the skin.

In addition, the composition of the present invention can be used in conjunction with other topically applied analgesic and/or systemically available agents for the treatment of acne.

Examples of such analgesic agents include, but are not limited to: morphine, cyclazocine, piperidine, piperazine, pyrrolidine, morphiceptin, meperidine, trifluadom, benzeneacetamine, diacylacetamide, benzomorphan, alkaloids, peptides, phenantrene and pharmaceutically acceptable salts, prodrugs or derivatives thereof. Specific examples of compounds contemplated by as suitable in the present invention include, but are not limited to morphine, heroin, hydromorphone, oxymorphone, levophanol, methadone, meperidine, fentanyl, codeine, hydrocodone, oxycodone, propoxyphene, buprenorphine, butorphanol, pentazocine and nalbuphine. As used in the context of opioid agents herein, “pharmaceutically acceptable salts, prodrugs and derivatives” refers to derivatives of the opioid analgesic compounds that are modified by, e.g., making acid or base salts thereof, or by modifying functional groups present on the compounds in such a way that the modifications are cleaved, either in routine manipulation or in vivo, to produce the analgesically active parent compound. Examples include but are not limited to mineral or organic salts of acidic residues such as amines, alkali or organic salts of acidic residues such as carboxylic acids, acetate, formate, sulfate, tartrate and benzoate derivatives, etc. Suitable opioid analgesic agents, including those specifically mentioned above, are also described in Goodman and Gilman, ibid, chapter 28, pp. 521-555.

Examples of systemically available agents which may be used in conjunction with the present compositions for the treatment of acne include, but are not limited to: retinoids such as tretinoin, isotretinoin, motretinide, adapalene, tazarotene, azelaic acid, and retinol; salicylic acid; resorcinol; sulfacetamide; urea; imidazoles such as ketoconazole and elubiol; essential oils; alpha-bisabolol; dipotassium glycyrrhizinate; camphor; beta.-glucan; allantoin; feverfew; flavonoids such as soy isoflavones; saw palmetto; chelating agents such as EDTA; lipase inhibitors such as silver and copper ions; hydrolyzed vegetable proteins; inorganic ions of chloride, iodide, fluoride, and their nonionic derivatives chlorine, iodine, fluorine; synthetic phospholipids and natural phospholipids; steroidal anti-inflammatory agents such as hydrocortisone, hydroxyltriamcinolone alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, fluticasone monopropionate, fluticasone furoate, mometasone furoate, budesonide, ciclesonide and salts are prodrugs thereof; nonsteroidal anti-Inflammatory drugs (NSAIDs) such as COX inhibitors, LOX inhibitors, p38 kinase inhibitors including ibuprofen, naproxen, salicylic acid, ketoprofen, hetprofen and diclofenac; analgesic active agents for treating pain and itch such as methyl salicylate, menthol, trolamine salicylate, capsaicin, lidocaine, benzocaine, pramoxine hydrochloride, and hydrocortisone; antibiotic agents such as mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, tetracycline, clindamycin, erythromycin; immunosuppressant agents such as cyclosporin and cytokine synthesis inhibitors, tetracycline, minocycline, and doxycycline, or any combination thereof.

In addition, other active agents may be included in the composition of the present invention, e.g., topically-effective anaesthetics such as xylocaine, cocaine, lidocaine, benzocaine, etc., which may provide a more immediate, if less effective in the long run, level of pain relief until the analgesic agent becomes fully effective.

Still other agents can also be administered, preferably topically, to potentiate the effects of the topically-administered cannabidiol. For example, dextromethorphan, a non-addictive opioid compound, can be co-administered, preferably topically, although parenteral administration is also effective, to enhance the effectiveness of the topically administered agent. Without wishing to be bound by theory, it is believed that dextromethorphan has previously unappreciated analgesic properties in peripheral nerves. Suitable concentrations of dextromethorphan are routinely ascertainable by the skilled worker, and include the normal therapeutic amounts administered parenterally for conventional purposes, e.g., as a cough suppressant, or less, and routinely determinable amounts for topical administration; for example, 1 g of dextromethorphan can be added to a composition disclosed herein to provide additional treatment for acne.

In one embodiment, the pharmaceutical composition of the present invention further comprises one or more of the following agents for the treatment of acne: retinoids such as tretinoin, isotretinoin, motretinide, adapalene, tazarotene, azelaic acid, and retinol; salicylic acid; resorcinol; sulfacetamide; urea; imidazoles such as ketoconazole and elubiol; essential oils; alpha-bisabolol; dipotassium glycyrrhizinate; camphor; beta.-glucan; allantoin; feverfew; flavonoids such as soy isoflavones; saw palmetto; chelating agents such as EDTA; lipase inhibitors such as silver and copper ions; hydrolyzed vegetable proteins; inorganic ions of chloride, iodide, fluoride, and their nonionic derivatives chlorine, iodine, fluorine; synthetic phospholipids and natural phospholipids; steroidal anti-inflammatory agents such as hydrocortisone, hydroxyltriamcinolone alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone, fluticasone monopropionate, fluticasone furoate, mometasone furoate, budesonide, ciclesonide and salts are prodrugs thereof; nonsteroidal anti-Inflammatory drugs (NSAIDs) such as COX inhibitors, LOX inhibitors, p38 kinase inhibitors including ibuprofen, naproxen, salicylic acid, ketoprofen, hetprofen and diclofenac; analgesic active agents for treating pain and itch such as methyl salicylate, menthol, trolamine salicylate, capsaicin, lidocaine, benzocaine, pramoxine hydrochloride, and hydrocortisone; antibiotic agents such as mupirocin, neomycin sulfate bacitracin, polymyxin B, 1-ofloxacin, clindamycin phosphate, gentamicin sulfate, metronidazole, hexylresorcinol, methylbenzethonium chloride, phenol, quaternary ammonium compounds, tea tree oil, tetracycline, clindamycin, erythromycin; immunosuppressant agents such as cyclosporine and cytokine synthesis inhibitors, tetracycline, minocycline, and doxycycline, or any combination thereof.

In preferred forms of the invention, the formulation is not a solid formulation, such as a patch or adhesive bandage. In preferred forms of the invention, the composition is a liquid formulation.

It is preferable that the composition concentrates the cannabinoid on the skin. To achieve this, one preferred method is to provide the cannabinoid in a composition comprising a mixture of a volatile solvent and a residual (less volatile) solvent.

### Volatile Solvents

By using a volatile solvent, one can achieve much higher, non-crystalline (i.e., in solution), concentrations of cannabinoids. The cannabinoids can be dissolved in much higher concentrations of the volatile solvent, and then once applied to the skin and the volatile solvent has evaporated, the cannabinoids remain on the skin in high concentrations. The volatile solvent may, for example, be a C₂₋₆ low molecular weight alcohol such as methanol, isopropanol, propanol, 2-butanol, n-butanol and ethanol. Alternatively, the volatile solvent may be a siloxane. Other suitable volatile solvents will be clear to the skilled reader.

In a preferred form of the invention, the composition comprises a combination of a C₂₋₆ low molecular weight alcohol and a siloxane.

Advantageously, in some embodiments, the volatile solvent is a liquid at ambient temperatures. Preferably the volatile solvent is liquid at about 30° C., or less, or at about 25° C. Preferably the level of volatility of the volatile solvent is about the same as that of isopropyl alcohol. Preferably, the boiling point of the volatile solvent is between about 70° C. and 110° C. at atmospheric pressure. Preferably, the boiling point of the volatile solvent is between about 80° C. and 105° C. at atmospheric pressure. Preferably, the boiling point of the volatile solvent is between about 85° C. and 105° C. at atmospheric pressure.

Advantageously, in some embodiments, the volatile solvent is selected from the group consisting of: C₂₋₆ alcohols, and combinations thereof. Advantageously, in some embodiments, the volatile solvent is selected from the group consisting of: C₂₋₄ alcohols, and combinations thereof. In specific embodiments, the volatile solvent is selected from the group consisting of: ethyl alcohol (or ethanol), n-propanol, isopropyl alcohol, butanol, and combinations thereof. Other volatile solvents will be clear to the skilled reader.

Alternatively, the volatile solvent comprises a siloxane. Preferably, the volatile solvent comprises a non-polymeric siloxane.

In a preferred form of the invention, the siloxane contains from one to eight silicon atoms per molecule. In a preferred form of the invention, the siloxane contains from two to five silicon atoms per molecule. In one embodiment, the siloxane contains two or three silicon atoms.

The siloxanes may have between one and eight methyl groups. In one embodiment, the siloxane is selected from the group consisting of: hexamethyldisiloxane, octamethyltrisiloxane and combinations thereof. These are the most volatile siloxanes, and are thus the most advantageous. Preferably the level of volatility of the siloxane is about the same as that of isopropyl alcohol.

In another embodiment, the siloxane contains 4 or 5 silicon atoms, and is, for example, decamethyltetrasiloxane or dodecamethylpentasiloxane. In another embodiment, the siloxane is a cyclical 4 or 5 silicon atom compound such octamethylcyclotetrasiloxane (CAS#556-67-2) or decamethylcyclopentasiloxane (CAS#541-C₂₋₆).

In one form of the invention, the volatile solvent is hexylmethyldisiloxane which is combined with less volatile polymethylsiloxane.

In a preferred form of the invention, the composition comprises a combination of a C₂₋₆ low molecular weight alcohol and a non-polymeric siloxane.

In a preferred form of the invention, the cannabinoid is dissolved in the volatile solvent.

In specific embodiments, the relative amount of volatile solvent is selected from the following group: at least 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, 15% w/w, 20% w/w, 25% w/w, 30% w/w, 35% w/w, 40% w/w, 45% w/w, 50% w/w, 55% w/w, 60% w/w, 65% w/w, 70% w/w, 75% w/w, 80% w/w, 85% w/w, 90% w/w, 95% w/w or 97% w/w. In specific embodiments, the maximum concentration of the volatile solvent is 50% w/w, 60% w/w, 70% w/w, 80% w/w, 90% w/w, 95% w/w or 97% w/w. The relative amount of volatile solvent may be between 1% w/w and 97% w/w, 10% w/w and 97%, 10% w/w and 90% w/w, 50% w/w and 97% w/w, 50% w/w and 95% w/w.

Preferably, the volatile solvent is provided as 85-95% w/w non-polymeric siloxane and 1-10% wt/wt C₂-C₆ alcohol.

Residual Solvents

The cannabinoids are preferably kept in a non-crystalline form on the skin after evaporation of the volatile solvent by the addition of a less volatile solvent. This less volatile solvent is called the residual solvent, as it may remain on the skin after evaporation of the volatile solvent to keep the cannabinoid in a non-crystalline state after evaporation of the volatile solvent. Preferably the residual solvent has a low volatility such that less than 5% would evaporate at skin temperature over 24 hours. Preferably, the residual solvent has a chain structure that has a hydrophobic end and a hydrophilic end. Preferably the residual solvent is a liquid at or below 32° C. Preferably the residual solvent dissolves the volatile solvent. Preferably the residual solvent maintains the cannabinoid in non-crystalline form, i.e. in solution, at concentrations of 20% up to 70% w/w cannabinoid.

The purpose of the residual solvent is to act as a solvent for the cannabinoid once the volatile solvent has evaporated. The residual solvent may be a compound from the list comprising: fatty acids, fatty acid alcohols, fatty alcohols, glycols or alkanes, or ethers of any of these. It is preferably a C₁₂₋₂₂ compound. The residual solvent may comprise a mixture of, for example, alkyl polypropylene glycol/polyethylene glycol ether and/or a fatty acid alcohol and/or a fatty alcohol. In specific embodiments the residual solvent is a C₁₂₋₂₂ fatty alcohol. In specific embodiments, the residual solvent is a C₁₆₋₂₂ fatty alcohol. In specific embodiments, the residual solvent is selected from the group consisting of: oleyl alcohol, isostearyl alcohol, isohexadecane, octyldodecyl alcohol, 2-hexyl decyl alcohol. Most preferably the residual solvent is isohexadecane.

In specific embodiments, the relative amount of residual solvent may be selected from the following group: at least 1% w/w, at least 2% w/w, at least 3% w/w, at least 4% w/w, at least 5% w/w, at least 6% w/w, at least 7% w/w, at least 8% w/w, at least 9% w/w, at least 10% w/w, at least 20% w/w, at least 30% w/w, at least 40% w/w, at least 50% w/w. In specific embodiments, the maximum concentration of the residual solvent is 50% w/w. In specific embodiments, the maximum concentration of the residual solvent is 80% w/w. The relative amount of residual solvent may be selected from the following group: between 1% and 80% w/w, between 1% and 50% w/w, between 1% and 40% w/w, between 1% and 30% w/w, between 1% and 20% w/w, between 1% and 10% w/w, between 2% and 80% w/w, between 2% and 50% w/w, between 2% and 20% w/w, between 2% and 10% w/w. Preferably the amount of residual solvent is between 1-10% w/w.

Preferably the amount of residual solvent is sufficient to keep the cannabinoid in a non-crystalline form, i.e. in solution, on the skin after partial or complete evaporation of the more volatile solvent or solvents.

Where the composition comprises a residual solvent and a volatile solvent, the composition comprises a solution of the cannabinoid in the mixture of the volatile solvent and the residual solvent. The composition may consist of a solution of the cannabinoid in the mixture of the volatile solvent and the residual solvent, or comprise a solution of the cannabinoid in the mixture of the volatile solvent and the residual solvent in combination with solid cannabinoid, such as a suspension of solid cannabinoid in a saturated solution of the cannabinoid in the mixture of volatile solvent and residual solvent. In preferred forms of the invention, the composition does not comprise solid cannabinoid.

The total amount of the volatile solvent, and the residual solvent if present, required is sufficient to keep the cannabinoid non-crystalline, i.e. in solution, at room temperature for between about 2-8 hours once the composition is applied to the skin.

The preferred ratio of cannabinoid to siloxane to residual solvent is selected from the range consisting of (w/w %):
between 0.5-20% cannabinoid, between 1-99% siloxane and between 0.1-98.5% residual solvent;
between 5-20% cannabinoid, between 4-70% siloxane and between 1%-70% residual solvent;
between 1-10% cannabinoid, between 20-98% siloxane and between 1-10% residual solvent.

The preferred ratio of cannabinoid to hexamethyldisiloxane to residual solvent is selected from the range consisting of (w/w %):
between 0.5-20% cannabinoid, between 1-99% hexamethyldisiloxane and between 0.1-98.5% residual solvent;
between 5-20% cannabinoid, between 4-70% hexamethyldisiloxane and between 1%-70% residual solvent;
between 1-10% cannabinoid, between 20-98% hexamethyldisiloxane and between 1-10% residual solvent.

As noted above, in highly preferred forms of the invention, the composition comprises 2.5% w/w of cannabidiol or 5% w/w of cannabidiol.

Where the composition contains 2.5% w/w or 5% w/w cannabidiol, the composition preferably comprises 85-95% w/w volatile solvent in the form of a non-polymeric siloxane. In a preferred form of the invention, the non-polymeric siloxane comprises two to three silicon atoms per molecule. In a preferred form of the invention, the non-polymeric siloxane is hexamethyldisiloxane.

In a preferred form of the invention, the viscosity of the siloxane, preferably hexamethyldisiloxane, is between 0.5 and 0.7 cSt.

Where the composition contains 2.5% w/w or 5% w/w cannabidiol and 85-95% w/w volatile solvent in the form of a non-polymeric siloxane, the composition optionally further comprises a volatile solvent in the form of a C₂₋₆ low molecular weight alcohol at a concentration of 1-10% w/w. In preferred forms of the invention, the concentration is 15% w/w. In preferred forms of the invention, the concentration is 2-4% w/w. In a preferred form of the invention, the C₂₋₆ low molecular weight alcohol is an alcohol containing between two and four carbon atoms per molecule. In preferred forms of the invention, the C₂₋₆ low molecular weight alcohol is isopropyl alcohol.

Where the composition contains 2.5% w/w or 5% w/w cannabidiol, 85-95% w/w volatile solvent in the form of a non-polymeric siloxane, and 1-10% w/w volatile solvent in the form of a C₂₋₆ low molecular weight alcohol, the composition optionally further comprises 1-10% w/w residual solvent in the form of fatty acids, fatty acid alcohols, fatty alcohols, glycols, alkanes, ethers of any of these, and combinations thereof. In a preferred form of the invention, the residual solvent is isohexadecane.

Viscosity Modifier

The present invention may include a viscosity modifier. The viscosity modifier has little effect on the delivery of the active cannabinoid from the composition, but may contribute significantly to patient compliance by improving the tactile qualities of the composition.

In one form of the invention, the viscosity modifier is a silicone fluid. In one form of the invention, the viscosity modifier is a polysiloxane. Where the viscosity modifier is a polysiloxane, the viscosity modifier is preferably a polydimethylsiloxane. Preferably, where the viscosity modifier is a polysiloxane, including a polydimethylsiloxane, the viscosity modifier has a viscosity of between 10,000 and 15,000 cSt, preferably still 11,500 and 13,500 cSt. In a highly preferred form of the invention, the viscosity modifier has a viscosity of approximately 12,500 cSt.

Where the polysiloxane viscosity modifier has a viscosity of between 10,000 and 15,000 cSt, the concentration of the polysiloxane viscosity modifier is preferably between 0.2 and 2% w/w. Preferably still, the concentration of the polysiloxane viscosity modifier is between 0.5 and 1.5% w/w. Preferably still, the concentration of the polysiloxane viscosity modifier is between 0.8 and 1.2% w/w.

The polysiloxane viscosity modifier may be provided in the form of a dimethiconol gum. The dimethiconol gum may be used alone, or in conjunction with another polysiloxane viscosity modifier, such as polydimethylsiloxane. In preferred forms of the invention, the dimethiconol gum is used in conjunction with the polydimethylsiloxane viscosity modifier. Preferably, the concentration of the dimethiconol gum viscosity modifier in the composition is between 3 and 7% w/w. Preferably, the concentration of the dimethiconol gum viscosity modifier in the composition is between 4 and 6% w/w. Preferably, the concentration of the dimethiconol gum viscosity modifier in the composition is between 4.5 and 5.5% w/w.

Such administration is expected to result in enhanced delivery of a cannabinoid, such as cannabidiol, to the epidermis and dermis of the skin, which is expected to be effective in significantly reducing, and therefore treating, acne in patients in need of such treatment.

In one preferred embodiment, the composition is non-aqueous. In another preferred embodiment, the composition does not comprise a preservative.

General

Throughout this specification, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Those skilled in the art will appreciate that the invention described herein is susceptible to variations and modifications other than those specifically described. The invention includes all such variation and modifications. The invention also includes all of the steps, features, compositions and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The invention described herein may include one or more range of values (e.g. concentration). A range of values will be understood to include all values within the range, including the values defining the range, and values adjacent to the range which lead to the same or substantially the same outcome as the values immediately adjacent to that value which defines the boundary to the range.

### EXAMPLES

Further features of the present invention are more fully described in the following description of several non-limiting embodiments thereof. The following Examples are to be construed as merely illustrative and not limitative of the remainder of the disclosure in any way whatsoever. This description is included solely for the purposes of exemplifying the present invention. It should not be understood as a restriction on the broad summary, disclosure or description of the invention as set out above.

### Example 1

### Techniques for Ascertaining Permeability of Compositions Containing Cannabidiol (CBD)

Dermatomed skin from a single donor was mounted in a Franz-type diffusion cell (0.55 cm² receptor fluid exposure surface area) and dosed with 5 ul of 2-[3-methyl-6-(1-methylethenyl)-2-cyclohexen-1-yl]-5-pentyl-1,3-benzenediol (CBD), BTX 1503 5% Solution, formulated in an mixture of a volatile solvent (hexylmethyldisiloxane/polymethylsiloxane-93% w/w), and residual solvent (arlamol E-2% w/w) at a concentration of 5.0% (w/w; 35.5 mg/ml). Following dosing, receptor phase samples were collected at 4, 10, 24 and 48 hours; after which the study was terminated.

The residual formulation was removed by tape stripping and the epidermis and dermis separated by blunt dissection. The levels of CBD in the epidermis, dermis, and receptor fluid samples were then analyzed using a bioanalytical method with LC-MS/MS detection.

The data showed that skin permeation (i.e., permeation through to the receptor phase of the test system) was negligible, with less than 0.081% (278 ng/cm²) in the receptor phase over the 48-hour exposure period.

The various layers of the skin showed different amounts of absorbed dose over the 48-hour period: epidermal deposition of CBD was 13.17% of the applied dose, while dermal deposition of CBD was 4.54% of the applied dose. The dermis concentration was 8,408 ng/cm² or 1,933 ng/g of tissue (1,933 ng/mL) following application of CBD mixture.

These results suggest that the level of systemic exposure for CBD is likely to be very low following topical administration in vivo.

### Example 2

The pharmacokinetics (PK) of single and multiple-dose administration of BTX 1503 5% Solution were evaluated in a healthy volunteer study. In this study, BTX 1503 5% Solution was applied as a single dose either QD or BID (12 hrs apart) on Day 1 followed by a 6-day washout period, then either QD or BID for 14 days (Day 8 to Day 21). Five subjects were enrolled in each cohort and doses were escalated for each sequential cohort enrolled with the following doses.
Cohort 1: 37.5 mg CBD/day or 0.066 mg/cm²/days applied as 1 mL of BTX 1503 5% (w/w) QD
Cohort 2: 75 mg CBD/day or 0.133 mg/cm²/day applied as 1 mL BTX 1503 5% (w/w) BID
Cohort 3: 112.5 mg CBD/day or 0.199 mg/cm²/day applied as 3 mL of BTX 1503 5% (w/w) QD
Cohort 4: 225 mg CBD/day or 0.398 mg/cm²/day applied as 3 mL of BTX 1503 5% (w/w) BID

Area of application assumed to be 565 cm² (i.e., on the face), which is reported by the European Union Scientific Committee on Consumer Safety (SCCS) to be half of the surface area for a female head (SCCS Notes of Guidance for the Testing of Cosmetic Substances and Their Safety Evaluation, 8th edition, 2012; Table 2).

Blood samples were taken for PK assessments on Day 1 (Baseline) at pre-dose (15 mins before dosing), 30, 60 and 90 mins and 2, 2.5, 3, 4, 6, 8 and 12 hrs, and 24 hrs after the first single dose. For participants that received BID dosing, samples were also taken at 30, 60 and 90 mins and 2, 2.5, 3, 4, 6, and 8 hrs after the second dose on Day 1.

During the multiple dose (14-day) phase, trough levels were obtained before the morning application on Day 15. On Day 21, blood samples were taken for PK assessments at pre-dose (15 mins before dosing), 30, 60 and 90 mins and 2, 2.5, 3, 4, 6, 8 and 12 hrs, 24 hrs and 48 hrs after the morning dose. For participants that received BID dosing, samples were also be taken at 30, 60 and 90 mins and 2, 2.5, 3, 4, 6, and 8 hrs after the second dose on Day 21. A sample was obtained on Day 23, 48 hrs after the last morning dose.

The PK after a single dose of BTX 1503 5% Solution showed that increased dosing (volume and frequency) resulted in increased plasma levels of CBD. CBD levels were first observed between 2 and 3 hrs after initial dosing. The mean Cmax after the first dose (QD or BID) was 0.309 ng/mL, 0.562 ng/mL, 0.626 ng/mL, and 0.876 ng/mL for Cohort 1, 2, 3, and 4, respectively. Tmax appears to occur at 12 hrs after QD dosing and at 18-20 hrs after BID dosing. Levels of CBD were below the limits of quantitation (BLOC); <0.2 ng/mL) for all cohorts by study Day 8, seven days after the initial dosing.

During the multiple dose phase of the study, mean trough levels on Day 15 did not show a clear dose effect. Day 15 plasma levels were not obtained for Cohort 1. The mean CBD trough level for Cohort 2 was 0.781 ng/mL, Cohort 3 was 0.525 ng/mL, and Cohort 4 was 2.11 ng/mL. There was one outlier in Cohort 4 that significantly skewed the mean levels (5.99 ng/mL). Without this subject, the mean trough level on Day 15 for Cohort 4 was 1.16 ng/m L.

By Day 21, CBD levels appeared to be at steady state as the second daily dose in the BID cohorts, Cohort 2 and Cohort 4, did not meaningfully elevate the CBD levels. In addition, the mean pre-dose levels on Day 21 (0.545, 0.770, 0.715, and 1.553 ng/mL) for each cohort, respectively, were not elevated above the Day 15 trough levels. The Cmax for Day 21 was a mean of 1.92 times the Day 1 Cmax (range 1.49 to 2.30) indicating that there was limited accumulation. CBD plasma levels drop dramatically between 24-48 hours after the final dose, but do not return to zero.

### Example 3

An Open-Label Study to Evaluate the Safety and Tolerability of BTX 1503 Solution in Patients with Acne Vulgaris

### Methodology:

Number of Subjects: 21 subjects enrolled; 18 completed the study. This was an open-label, single-arm study.

### Diagnosis and Main Criteria for Inclusion:

This study included males and females between 18 and 65 years of age (inclusive). Subjects were in good general health without clinically significant disease and had acne vulgaris of the face with 20 to 50 (inclusive) inflammatory lesions on the face, 20 to 100 (inclusive) non-inflammatory lesions on the face, an Investigator Global Assessment (IGA) score for acne severity of 3 or 4 (moderate or severe) assessed on the face and 3 nodular/cystic acne lesions (>5 mm in diameter).

To ensure the validity of the clinical assessments, subjects were instructed to use only the study provided cleanser (Cetaphil) on the face throughout the study. The face was washed daily with this cleanser during the subject's normal daily routine of care. Cleansing or shaving of the face was prohibited within 5 minutes prior to study drug application so as not to interfere with cutaneous tolerability assessments. The face was not to be washed within 4 hours after study drug application. In addition, cleansing, shaving, swimming, heavy exercise, or application of sunscreens was prohibited for 4 hours after application of study drug to maximize the time allowed for study drug absorption. Subjects agreed to maintain their regular use of sunscreens, moisturizers, and facial makeup throughout the entire course of the study and not apply sunscreens, moisturizers, or facial makeup within 4 hours prior to, or 1 hour after, study drug application.

Subjects were also instructed to avoid excessive ultraviolet radiation exposure as might be experienced while sunbathing or tanning. Hats, sunglasses, and other protective garments were to be worn to protect the area treated with study drug throughout the study.

Throughout the study, every attempt was made to keep the individual use of concomitant therapies consistent. Medications that could have interfered with the efficacy and/or safety assessments were prohibited.

### Test Product, Dose and Mode of Administration, Batch Number:

### Administration:

BTX 1503 5% (w/w) Solution: each dose consisted of 3 mL of the study drug applied topically to the face twice (BID) daily (at about the same time each day) using an applicator swab. Each milliliter of BTX 1503 5% (w/w) solution contains 37.5 mg of CBD. Therefore, all subjects received 225 mg/day of CBD. The drug product contains 5% (w/w) concentration of CBD in a formulation of excipients which have been used in other topical products. The solution spreads easily and evaporates quickly leaving the CBD and a small amount of the excipients on the skin.

### Selection of Doses in the Study

The maximum feasible concentration of 5.0% (w/w) BTX 1503 Solution was safe for testing on the skin based on a completed Phase 1a clinical study (BTX.2017.001) with BTX 1503 5% Solution in Australia in accordance with ICH GCPs. In this study, the highest dose of 225 mg CBD/day or 0.398 mg/cm²/day applied as 3 mL of BTX 1503 5% (w/w) BID was considered safe based on safety, tolerability, and PK outcomes in healthy volunteers following 14 consecutive days of dosing.

In this study, patients with moderate to severe acne received 225 mg/day of CBD (0.398 mg/cm²/day, or 3.75 mg/kg/day) for 28 consecutive days. This dose level is well below that tested and shown to be well-tolerated in a 28-day study in minipigs. Specifically, the NOAEL for dermal tolerability of BTX 1503 5% (w/w) on the skin of minipigs was 3.0 mg/cm²/day (150 mg/kg/day), which is ˜7.5 times the daily dose delivered in this study. In addition, based on the ratio of the mean Cmax observed in the 28-day minipig study to the mean Cₘₐₓ in the 3 mL BID cohort in the Phase 1a healthy volunteer study, there was >300 times the level of CBD, with no observed effect, in the minipigs.

Therefore, the dose level used in this study was lower or identical to that previously shown to be well-tolerated in both nonclinical and clinical studies for BTX 1503 5% Solution.

The first application of study drug was applied by the clinical site staff. Cutaneous tolerability was assessed prior to and 1 hour after application. A diary to collect dosing compliance and study drug was dispensed at the Baseline Visit.

### Duration of Treatment:

28 days, twice daily on Days 1 through 27 and once on Day 28 for a total of 55 doses

At the Screening Visit, informed consent, medical history, demographics, vital signs, height and weight, tobacco and alcohol history, and a urine pregnancy test (UPT) for women of child bearing potential (WOCBP) were obtained. A urine drug screen (UDS) was performed. In addition, lesion counts on the face and an Investigator's Global Assessment (IGA) were conducted to assess subject eligibility.

Eligible subjects were enrolled within 14 days after the Screening Visit. Assessments for safety (CBC, chemistry, urinalysis, and vital signs) were obtained at the Baseline Visit (Day 1). If the Screening and Baseline Visits were not conducted on the same day, a UPT for WOCBP, lesion counts on the face, an Investigator's Global Assessment (IGA) for facial acne and a UDS were repeated. Baseline photographs of the face and a blood sample for Baseline study drug plasma levels were obtained. Clinical site staff applied the first dose of study drug and subjects were observed in the clinic for one hour after application on Day 1. Cutaneous tolerability assessments were conducted at one hour after the first application. Subjects were given two weeks of study drug and instructed in the proper application to cover their entire face twice daily.

On Day 7, a call was made to each subject to ensure that they continued with dosing per instructions.

Subjects returned to the clinic on Day 14 for vital signs, cutaneous tolerability assessments, and a blood draw for study drug plasma levels. Subjects were also queried for adverse events (AEs) and changes in concomitant medications. Diaries and study drug were returned and reviewed for compliance. In addition, the subject applied their morning dose of study drug during the visit for the clinical site to confirm correct application techniques. Another 14 days of study drug were dispensed along with the diary for the last two weeks of study drug treatment.

Subjects returned to the clinic on Day 28 for safety assessments; vital signs, AEs, blood samples for CBC, chemistry and drug levels, and urine sample for urinalysis and urine drug test. A UPT was conducted for WOCBP. Cutaneous tolerability assessments were also obtained. Lesion counts on the face and an IGA for facial acne were conducted. Photographs of the face were obtained, and the patient reported outcome (PRO) was administered.

Subjects returned to the clinic one week following their final dose (Day 35). Safety labs were obtained if abnormal at Day 28. A plasma sample for study drug levels was obtained, cutaneous tolerability assessments were obtained and AEs were reviewed. Lesion counts on the face and an IGA for facial acne were conducted and photographs of the face were obtained.

TABLE 1
Investigator's Global Assessment Scale for Acne Vulgaris

| Grade | Description |
|---|---|
| 0 | Clear skin with no inflammatory or non-inflammatory lesions |
| 1 | Almost clear; rare non-inflammatory lesions with no more than |
| | one small inflammatory lesion |
| 2 | Mild severity; greater than Grade 1; some noninflammatory |
| | lesions with no more than a few inflammatory lesions (papules/ |
| | pustules only, no nodular lesions) |
| 3 | Moderate severity; greater than Grade 2; up to many non- |
| | inflammatory lesions and may have some inflammatory lesions, |
| | but no more than one small nodular lesion |
| 4 | Severe; greater than Grade 3; up to many non-inflammatory and |
| | inflammatory lesions, but no more than a few (≤3) nodular |
| | lesions |

Photographs of the subject's face were obtained at the Baseline Visit (Day 1), Day 28 Visit, and the Day 35 Visit. These photographs were reviewed and assessed for IGA scores by an independent review panel (IPR) at the completion of the study to assess inter-rater variability for future study designs.

On Day 28, the subject was asked to complete the Patient Reported Outcome (PRO) to assess the perception of their acne relative to their baseline. The subject completed the assessment to answer the following question: “Compared to the beginning of treatment, my acne is?” with a response of “Much better”, “Slightly better”, “The same”, “Slightly worse”, or “Much worse”. Lesion Counts

Inflammatory and non-inflammatory counts (counted separately) were collected at Screening/Baseline, Day 28 and Day 35.

Inflammatory, non-inflammatory, and total lesion counts were summarised and listed. All data collected at scheduled and unscheduled visits was included in the listings.

The absolute changes and percentage changes from Baseline to each post-Baseline visit values were calculated for inflammatory, non-inflammatory and total lesion counts.

The summary of lesion counts table presents summary statistics for the results and the absolute change and percentage change from Baseline values at each scheduled post-Baseline visit. The two-sided 95% CI for the mean was presented for all mean values. In addition, the absolute and percentage change from Baseline values were analyzed using a paired t-test to test the hypothesis that there was a reduction in the lesion counts compared to Baseline. The corresponding one-sided p-value were presented.

A figure of the mean number of lesions ±the standard error of the mean (SE) over time was presented for each lesion type.

The listing of lesion counts includes all information (fields) that was collected on the Inflammatory Lesion Counts and Non-Inflammatory Lesion Counts eCRF pages. In addition, the observation that was used as the Baseline record (value) for each lesion count type was flagged, and the absolute changes and percentage changes from Baseline values at each post-Baseline visit were presented.

### Investigator's Global Assessment (IGA)

The IGA score (IGA-Investigator) was conducted at Screening/Baseline, Day 28 and Day 35.

The IGA score was based on the scoring outlined in Table 1.

The dichotomized IGA of success was defined as an IGA of ‘Clear’ (0) or ‘Almost Clear’ (1) and a minimum 2-grade improvement from Baseline at the specific post-Baseline visit. The response at each post-Baseline visit was derived based on the Investigator IGA scores.

Investigator IGA scores were summarised and listed. All data collected at scheduled and unscheduled visits were included in the listings.

The absolute change from Baseline to each post-Baseline visit values were calculated for the Investigator IGA scores.

The summary of IGA scores table presents the frequency distributions of the IGA scores (frequencies and percentages) for the Baseline and each scheduled post-Baseline visit score for each of the Investigator IGA scores. In addition, the dichotomized IGA response (success/failure) at each scheduled post-Baseline visit was summarized, and the 95% Clopper-Pearson CI for the success response rate presented. The proportion of success responses was also analyzed with a binomial test to test the hypothesis that the response rate is greater than 0%, and the corresponding one-sided p-value was presented.

The quantitative summary of IGA scores table presents summary statistics for the Investigator IGA scores, as well as the change from Baseline values at each scheduled post-Baseline visit. The change from Baseline values were analyzed using a Wilcoxon's signed-rank test to test the hypothesis that there was an improvement in the IGA scores compared to Baseline. The corresponding one-sided p-value was presented.

This shift from Baseline in IGA scores table presents frequencies and percentages for each Baseline score, as well as frequencies and percentages for the scheduled post-Baseline IGA scores within each of the Baseline scores (i.e., the shift from Baseline to post-Baseline).

The IGA scores over time was presented in stacked bar charts based on the percentage of subjects reporting each score at each visit.

The listings of IGA scores includes all the information (fields) that was collected on the Investigator's Global Assessment (IGA) eCRF pages. In addition, the observation that was used as the Baseline record (value) for each assessment was flagged, and the IGA response and change from Baseline value at each post-Baseline visit was presented.

Photographs of the subjects' faces were also obtained at the specified time points. These photographs were evaluated by an independent panel of dermatologists to determine the IGA central panel score (IGA-Central Panel). This information was provided electronically and was not captured on the eCRF. The same analyses that were applied to the Investigator IGA Scores were also applied to the Central Panel IGA Scores. Central Panel IGA scores were summarised and listed.

### Criteria for Evaluation:

### Safety and Tolerability

Safety was the primary outcome measure. The safety outcomes were measured through assessment of:
AEs monitored from time of consent through the end of study.
Cutaneous tolerability (erythema, scaling, dryness, burning/stinging, and irritant/allergic contact dermatitis) collected at Baseline, Day 14, Day 28, and Day 35 and graded using the following scale: 0, None; 1, Slight; 2, Moderate; 3, Severe.
Vital signs (temperature, blood pressure, and pulse) obtained at Baseline, Day 14, Day 28 and Day 35.
Complete blood count (CBC), chemistry, and urinalysis at Baseline and at Day 28.
   Blood levels of study drug will be measured at Baseline and prior to dosing (trough level) on Day 14, Day 28, and Day 35. Urine drug tests for drug of abuse were conducted at the Day 1, Day 28 and Day 35 Visits.

Pregnancy testing was conducted for WOCBP at the Screening Visit, the Day 1 visit (if >7 days from the Screening Visit), and at the Day 28 Visit.

### Pharmacological Activity

Assessments of pharmacological activity were evaluated by the treating dermatologist(s) through collection of lesion counts and Investigator Global Assessment (IGA) scores at Baseline, Day 28, and Day 35. Photographs were obtained at Baseline, Day 28, and Day 35. An independent group of dermatologists also reviewed the photographs for IGA scoring. On Day 28 a PRO instrument assessed the subject's perception of the change in their acne relative to baseline.

### Statistical Methods:

All statistical processing was performed using SAS® 9.4.

Two analysis populations were defined for this study, the safety and the pharmacology analysis populations (Pharmacology Population). The Safety Population is comprised of all enrolled subjects who received at least one application of BTX 1503 (full or partial application). Subjects who prematurely discontinue from the study were not excluded from the Safety Population. Furthermore, no subject was excluded from the Safety Population due to protocol deviations.

The Pharmacology Population is comprised of all enrolled subjects who were included in the Safety Population and have Day 28 or Day 35 lesion assessments or IGA scores. If a subject was missing one of the lesions counts, either inflammatory or non-inflammatory, the subject was included in the population, but no data was contributed to the summaries.

### Safety Analyses:

All treatment-emergent adverse events (TEAEs) occurring during the study were recorded and classified based on MedDRA terminology. Treatment-emergent adverse events were those AEs with an onset on or after the first application of study medication. All reported TEAEs were summarised by treatment group, the number of subjects reporting events, system organ class, preferred term, severity, relationship to study drug, and seriousness. When summarizing events by causality and severity, each subject was counted only once within a system organ class or a preferred term by using the event with the greatest relationship and highest severity within each classification.

Serious adverse events (SAEs) were summarised by system organ class, preferred term, severity, outcome and relationship to study drug; and all SAEs were listed by subject. In addition, a list of subjects who prematurely discontinued from the study due to an AE and the reason for discontinuation were provided.

Concomitant medications were mapped to ATC Level 2 using the WHODrug dictionary. The number and percentage of subjects reporting each medication were summarised. Medications taken by each subject were listed.

Cutaneous tolerability scores for each parameter (erythema, scaling, dryness, burning/stinging, and irritant/allergic contact dermatitis) were summarised for each visit. In addition, the change from baseline in the mean scores were summarised for each visit.

### Exploratory Analyses:

Lesion counts were collected by the clinical site along with photographs of the subject's face. Inflammatory and non-inflammatory lesion counts were made separately. The IGA was conducted by the study investigator at each site. Each subject was to have the IGA done by the same investigator throughout the study. Photographs of the subjects were obtained and IGA was also assessed by the central panel's review of photographs.

Demographics were summarised by age, gender, race, ethnicity height and weight. Summary statistics were prepared for the change from baseline in lesion counts (inflammatory and non-inflammatory separate and combined) and IGA separately for the investigators and the central panel (IGA only). For continuous variables, the mean, standard deviation (SD), median, and range were presented along with the 95% confidence interval (CI). Categorical variables were summarised by

### Demographics and Baseline Characteristics:

Twenty-one (21) subjects (Safety Population) were enrolled and ranged in age from 18 to 35 years, with a mean (SD) age of 23.3 (±6.30) years. There were more females (81%) than males. All subjects reported they were not Hispanic or Latino. Most subjects were White (76.2%), 14.3% were Asian, and 9.5% reported Other (Middle Eastern and Bangladesh). Baseline characteristics of height and weight were typical for the ages evaluated. The majority of subjects (66.7%) drank alcohol. Most subjects (95.2%) never smoked and one subject was a former smoker. The medical history of subjects was typical of an otherwise healthy population of patients with acne vulgaris.

The mean (±SD) number of inflammatory lesions at the Baseline Visit was 36.4 (±7.45). The mean (±SD) number of non-inflammatory lesions at the Baseline Visit was 35.9 (±16.98). Most subjects (77.8%) had moderate severity acne based on the IGA at the Baseline Visit.

Eighteen subjects completed the 28-day treatment (Pharmacology Population).

### Pharmacological Activity Results:

The pharmacological activity of treatment with 3 mL (112.5 mg) of CBD twice daily for 28 days was evaluated through analysis of changes from Baseline in inflammatory and non-inflammatory lesion counts, investigator and IPR assessed IGA, and a PRO evaluating the subjects' assessment of change from Baseline. All three of these assessments demonstrated that treatment with BTX 1503 5% (w/w) resulted in overall improvement in facial acne vulgaris.

Inflammatory lesion counts at Day 28 decreased 28.7% from Baseline for the Pharmacology Population with a 47.0% decrease in an appropriately executed sensitivity analysis where two outliers were removed. At Day 35, 7 days after completion of treatment, inflammatory lesion counts decreased further from Baseline at 37.5% for the Pharmacology Population and remained decreased at 45.2% for the sensitivity analysis.

Mean non-inflammatory lesion counts at Day 28 decreased 6.9% from Baseline for the Pharmacology Population and by 12.4% in the sensitivity analysis. At Day 35, 7 days after completion of treatment, mean non-inflammatory lesion counts decreased further from Baseline at 21.4% for the Pharmacology Population and 22.4% for the sensitivity analysis.

In this 28-day treatment study, IGA improved with 5 subjects (27.8%) having mild acne (IGA=2) at Day 28 and 6 subjects (33.3%) with mild acne at Day 35. Compared to baseline, 5 subjects (27.8%) achieved at least a 1-grade improvement in IGA at Day 28. There was 1 subject (5.6%) that achieved a 2-grade improvement in IGA at Day 28. IGA success defined as an IGA score of “Clear” or “Almost Clear” and a 2-point decrease from Baseline was not observed at Day 28 or Day 35 when the IGA was assessed by the study investigator. In the IPR analysis 2 subjects (12.5%) had an IGA success at Day 28.

For the PRO assessment, 9 subjects (50.0%) reported that their acne was Slightly Better (33.3%) or Much Better (16.7%) compared to start of treatment. Two subjects (11.1%) reported Slightly Worse and no subjects reported Much Worse.

Study drug (CBD) plasma levels were low throughout the study. Nine subjects still had circulating levels of CBD at the Day 35 visit, likely due to a depot effect of the CBD in the skin which eluted over time. The levels of CBD observed in this study are similar to those observed in a healthy volunteer study demonstrating that CBD is not more readily absorbed in subjects with acne vulgaris. There was no correlation between CBD plasma levels and the change from Baseline in inflammatory lesion counts (r²=0.079).

### Safety Results:

This study demonstrated that daily topical treatment with 3 mL BID of BTX 1503 5% Solution (225 mg CBD per day) was safe and well tolerated. There were no SAEs reported. No AEs resulted in discontinuation from the study or modification of study drug dosing.

Seven AEs were reported in 6 of the 21 subjects (28.6%). All AEs were reported as mild except one moderate, unrelated event of presyncope. Only one event of mild application site pain (sore eyes) was reported as possibly related. The other mild, unrelated AEs reported in one subject each were urinary tract infection, viral respiratory tract infection, presyncope, somnolence, and panic attack.

Slight to moderate erythema was reported most frequently in the cutaneous tolerability assessments. However, most subjects that reported erythema pre- or post-study drug application had erythema at Baseline and treatment with BTX 1503 did not exacerbate the erythema. Only one subject had increased erythema from Baseline and this was reported at Day 35, seven days after their final application of study drug. Slight burning/stinging was reported in 5 subjects (23.4%), Slight dryness was reported in 4 subjects (19.0%), and Slight scaling was reported in 2 subjects (9.5%). Only one positive cutaneous tolerability assessment (Slight dryness) was reported at more than a single visit.

There were no clinically relevant changes from baseline observed in safety laboratory assessments (CBC, chemistry, and urinalysis), or in vital signs (blood pressure, temperature and pulse). No subjects tested positive for the presence of THC using a urine drug test.

### Summary

In this study of 21 subjects with moderate to severe facial acne vulgaris, treatment with up to 28 days of topically applied BTX 1503 5% (w/w) (225 mg daily) was safe and well tolerated. Pharmacological activity of BTX 1503 was observed through statistically significant improvement from Baseline in inflammatory and non-inflammatory lesion counts. Improvements were also observed in the IGA and in the PRO, although the short study duration may have limited a more robust response. A sensitivity analysis was conducted to exclude 2 extreme outliers.

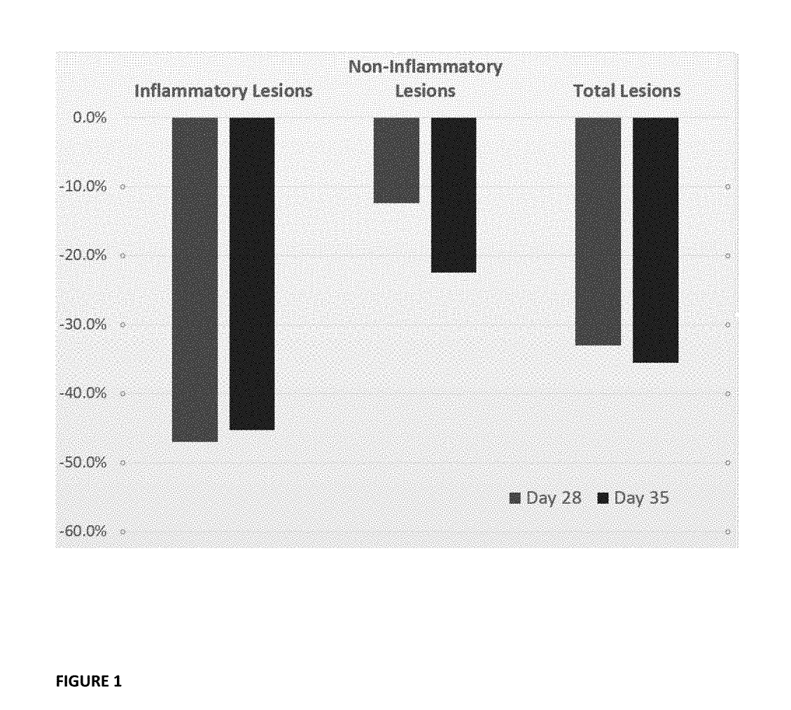

Compared to baseline, 5 subjects (27.8%) achieved at least a 1-grade improvement in IGA at Day 28. There was 1 subject (5.6%) that achieved a 2-grade improvement in IGA over the same time period. The percent changes in lesion counts are shown in FIG. 1.

Patient satisfaction was high with 9 of the 18 subjects (50.0%) reporting their acne improved (Slightly Better=33.3%, Much Better=16.7%) compared to the start of treatment.

Topical treatment with BTX 1503 for 28 days in patients with moderate to severe acne was safe, well tolerated and did not result in any significant skin irritation. Large, statistically significant reductions in inflammatory lesions were observed after 28 days that correlated with high overall patient satisfaction.

When compared to results in a healthy volunteer study which also studied up to 225 mg of CBD applied daily, treatment of subjects with acne vulgaris resulted in comparable or better safety and tolerability. No serious or severe AEs were reported and no subjects withdrew from the study due to an AE. AEs associated with the study drug were mild and only one AE (sore eyes) was reported as possibly related. Plasma levels of CDB were low and similar to those in healthy volunteers.

This open-label study supports the safety. Tolerability and pharmacological activity of CBD when used to treat subjects with acne vulgaris. Randomized, controlled, double-blind studies are needed to confirm the activity and demonstrate the efficacy and safety of 12 weeks of treatment with BTX 1503.

### Example 4

A Randomized, Double-Blinded, Vehicle-Controlled Study to Evaluate the Safety and Efficacy of BTX 1503 in Patients with Moderate to Severe Acne Vulgaris (Phase 2)

### Methodology:

Number of Subjects: 360 subjects. Subjects will be randomized 2:2:2:1:1 (BTX 1503 5% BID:BTX 1503 5% QD:BTX 1503 2.5% QD:Vehicle BID:Vehicle QD) with 90 subjects in each BTX 1503 group and 45 subjects in each vehicle group.

Each milliliter of the BTX 1503 5% liquid formulation contains 37.5 mg of CBD. Each milliliter of the BTX 1503 2.5% liquid formulation contains 18.75 mg of CBD. All subjects will apply 2.0 mL, 4 pump actuations, of BTX 1503 BID or QD or Vehicle BID or QD based on their randomized treatment group. Subjects will receive the following daily exposure to CBD.
Subjects randomized to BTX 1503 5% BID will apply 150.0 mg of CBD daily,
Subjects randomized to BTX 1503 5% QD will apply 75.0 mg of CBD daily,
Subjects randomized to BTX 1503 2.5% QD will apply 37.5 mg of CBD daily.

Study drug will be supplied in 60 mL multi-dose, metered pumps delivering 0.5 mL per actuation. Each pump for BID dosing will contain approximately 39 mL of study drug and each pump for QD dosing will contain approximately 21 mL of study drug. This will provide dosing for 7 days for all subjects. Pumps for all groups will be labelled identically, except for kit number and bottle number, to maintain the blind.

Study drug will be pumped into the palm of one hand and applied to the face using fingertips of the other hand. Study drug will be applied to the entire face, regardless of location of acne lesions.

### Diagnosis and Main Criteria for Inclusion:

This study will include males and females between 18 and 65 years of age (inclusive). Subjects will be in good general health without clinically significant disease and have:
acne vulgaris of the face with 20 to 50 (inclusive) inflammatory lesions on the face,
20 to 100 (inclusive) non-inflammatory lesions on the face,
an Investigator Global Assessment (IGA) score for acne severity of 3 or 4 (moderate or severe) assessed on the face, and
≤3 nodular/cystic acne lesions (>5 mm in diameter).

To ensure the validity of the clinical assessments, subjects will be instructed to use only the study provided cleanser (Cetaphil®) on the face throughout the study. Faces were washed daily with this cleanser during the subject's normal daily routine of care. Cleansing or shaving of the face is prohibited within 5 minutes prior to study drug application so as not to interfere with cutaneous tolerability assessments. The face is not to be washed within 4 hours after study drug application. In addition, cleansing, shaving, swimming, heavy exercise, or application of sunscreens is prohibited for 4 hours after application of study drug to maximize the time allowed for study drug absorption. Subjects must agree to maintain their regular use of sunscreens, moisturizers, and facial makeup throughout the entire course of the study and not apply sunscreens, moisturizers, or facial makeup within 4 hours prior to, or 1 hour after, study drug application.

### Administration:

Baseline photographs of the face (selected sites) will be obtained. The Acne-QoL will be administered. Clinical site staff will apply the first dose of study drug. Cutaneous tolerability assessments will be conducted prior to and approximately 15 minutes after the first application. Subjects will be given a diary and sufficient study drug to last until their Day 28 Visit and instructed in the proper application to cover their entire face.

Subjects will return to the clinic on Day 14 for a review of their diary to ensure compliance with study drug applications. Lesion counts, IGA and cutaneous tolerability assessments will be conducted. In addition, the subject will apply study drug during the visit for the clinical site to confirm correct application techniques. AEs and concomitant medications will be reviewed.

Subjects will return to the clinic on Day 28 and Day 56 for cutaneous tolerability assessments, lesion counts and IGA. Subjects will also be queried for AEs and changes in concomitant medications. Diaries and study drug will be returned and reviewed for compliance. In addition, the subject will apply study drug during the visit for the clinical site to confirm correct application techniques. Study drug will be dispensed along with the diary for the next 28 days of study drug treatment.

Subjects will return to the clinic for their final visit on Day 84 for safety, tolerability and efficacy assessments, including lesion counts and IGA scoring of facial acne. Safety labs (CBC, chemistry, and urinalysis) will be obtained. Photographs of the face will be obtained at selected sites. Cutaneous tolerability assessments will be conducted, and concomitant medications and AEs will be reviewed. The Acne-QoL and a patient reported outcome (PRO) will be administered at Day 84, assessing the subject's perception of the change in their acne relative to Baseline.

The study will be evaluated using 3 analysis sets: intent-to-treat (ITT), per protocol (PP), and safety. Efficacy conclusions will be drawn from the ITT analysis set. The PP analysis set will be used to support the efficacy findings in the ITT analyses. Safety conclusions will be drawn from the safety analysis set.

The efficacy analyses will be performed using the ITT (primary) and PP (supportive) analysis sets. The efficacy variables include the IGA and lesion counts (inflammatory and non-inflammatory) collected at Screening/Baseline and all subsequent study visits. The primary efficacy endpoint is the absolute change in inflammatory lesion count at Day 84.

Absolute and percent changes in lesion counts from Baseline will be calculated for each subject at Day 14, Day 28, Day 56 and Day 84. The IGA will be dichotomized into “success” and “failure” at study Day 14, Day 28, Day 56, and Day 84 with a subject considered a “success” at each individual visit if the IGA at that visit is Clear (“0”) or Almost Clear (“1”) and at least 2 grades less than the Baseline score. Exploratory efficacy assessments also include the Acne-QoL which will be scored according to the author's scoring system (Martin 2001), and the subject's assessment of improvement (PRO) using proportions by category.

Descriptive statistics (including mean, median, standard deviation [SD], minimum, and maximum, unless otherwise stated) will be presented for the following parameters by study group using both the ITT and PP analysis sets:

Inflammatory, non-inflammatory, and total lesion counts at Baseline, Day 14, Day 28, Day 56, and Day 84,

Absolute and percent change from Baseline in inflammatory, non-inflammatory, and total lesion counts at study Day 14, Day 28, Day 56, and Day 84,

IGA scores and frequency and percent distribution of the dichotomized IGA at study Day 14, Day 28, Day 56, and Day 84.

This Phase 2 study is designed to identify the response to two different dosing frequencies and two concentrations of BTX 1503. Statistical tests applied to the outcomes will be exploratory. No adjustments for Type 1 error will occur.

The change from Baseline in lesion counts (inflammatory and non-inflammatory; separate and combined, and total) at Days 14, 28, 56, and 84 will be analyzed using ANCOVA with Baseline lesion count and treatment as covariates.

Success on IGA defined as a score of clear or almost clear and/or at least a 2-grade improvement from Baseline at Day 14, Day 28, Day 56, and Day 84 will be analyzed using logistic regression, adjusting for Baseline IGA.

### Cutaneous Tolerability:

Cutaneous tolerability (erythema, scaling, dryness, pruritus, and burning/stinging) will be summarized by treatment group at the Baseline, Day 14, Day 28, Day 56, and Day 84 Visits. Cutaneous tolerability will be graded using the following scale: 0, None; 1, Slight; 2, Moderate; 3, Severe.

TABLE 2
Cutaneous Tolerability Assessments Scale for Acne Vulgaris

| Tolerability | Severity | | | |
|---|---|---|---|---|
| Assessment | None = 0 | Mild = 1 | Moderate = 2 | Severe = 3 |
| Erythema | No erythema | Slight pinkness | Definite | Intense |
| | | present | redness, easily | redness |
| | | | recognized | |
| Scaling | No scaling | Barely | Obvious but no | Heavy scale |
| | | perceptible | profuse | production |
| | | shedding, | shedding | |
| | | noticeable only | | |
| | | on light | | |
| | | scratching or | | |
| | | rubbing | | |
| Dryness | No dryness | Slight but | Moderate | Marked |
| | | definite | roughness | roughness |
| | | roughness | | |
| Pruritus (in last | No itching | Only aware of | Often aware of | Constant |
| 24 hours) | | itching at | itching; | itching; |
| | | times; only | annoying; | distressing; |
| | | present when | sometimes | frequent sleep |
| | | relaxing; not | disturbs sleep | disturbance; |
| | | present when | and daytime | interferes with |
| | | focused on | activities | activities |
| | | other activities | | |
| Burning/Stinging | No | Slight warm, | Definite warm, | Hot, |
| (in last 24 | Burning/Stinging | tingling/stinging | tingling/stinging | tingling/stinging |
| hours) | | sensation; not | sensation that | sensation that |
| | | really | is somewhat | has caused |
| | | bothersome | bothersome | definite |
| | | | | discomfort |

The following efficacy assessments will be performed at the Screening, Baseline, Day 14, Day 28, Day 56, and Day 84 Visits:
Counting of inflammatory and non-inflammatory lesions of the face by the principal investigator (PI) or appropriately trained designee. Thorough, documented, training will be provided to the PI and/or designee in the method for identifying and counting lesions.
Administration of the IGA by the PI or appropriately trained designee. The IGA will be graded based on the scale provided in Table 3.

TABLE 3
Investigator's Global Assessment Scale for Acne Vulgaris

| Grade | | Description |
|---|---|---|
| 0 | Clear | No evidence of facial acne vulgaris |
| 1 | Almost | Few non-inflammatory lesions (comedones) are present; |
| | Clear | a few non-inflamed papules (papules must be resolving |
| | | and may be hyperpigmented, though not pink-red) may |
| | | be present |
| 2 | Mild | Several to many non-inflammatory lesions (comedones) |
| | | are present; a few inflammatory lesions (papules/ |
| | | pustules) are present; no nodulo-cystic lesions |
| 3 | Moderate | Many non-inflammatory (comedones) and inflammatory |
| | | (papules/pustules) are present; there may or may not be |
| | | one small nodulo-cystic lesion |
| 4 | Severe | Significant degree of inflammatory disease; papules/ |
| | | pustules are a predominant feature; a few nodulo-cystic |
| | | lesions may be present; many comedones may be present. |

Photographs of the subject's face will be obtained at selected sites at the Baseline Visit and the Day 84 Visit. Details on the methods for photography are provided in the Photography Manual.
At Baseline and at Day 84, the Acne-QoL will be administered.
On Day 84, the subject will be asked to complete the Patient Reported Outcome (PRO) to assess the perception of their acne relative to their baseline. The subject will complete the assessment to answer the following question: “Compared to the beginning of treatment, my acne is?” with a response of “Much better”, “Slightly better”, “The same”, “Slightly Worse”, or “Much worse”.

For purposes of conducting IGA scoring, the following definitions will apply:
Open comedo—a widely dilated follicle, plugged with sebum and keratin (blackhead)
Closed comedo—a small, flesh-colored closed follicle, filled with sebum and firm to palpation
Papule—a small solid, inflamed, elevated lesion less than 5 mm in diameter
Pustule—a circumscribed, erythematous raised skin lesion containing white exudate or pus, less than 5 mm in diameter
Nodule—an erythematous, raised, firm, deep-seated papule equal to or greater than 5 mm in diameter

### Statistical and Analytical Plans

A separate Statistical Analysis Plan (SAP) will be prepared for this study. The statistical approaches to analysis of the data are described in this protocol. Further detail on the structure of tables, listings, and figures is provided in the SAP.

The purpose of this Phase 2 study is to describe the safety and efficacy of treatment with the BTX 1503 5% liquid formulation or 2.5% liquid formulation vs Vehicle liquid formulation with QD or BID dosing in subjects with acne vulgaris. P-values for selected variables will be presented to assist in evaluating the outcome of the study. Failure to achieve a statistically significant result does not imply a failed study; results from this study will be used to inform statistical approaches for registration studies.

The primary efficacy endpoint of the study is the change from Baseline in inflammatory lesion counts. The study will evaluate the superiority of active study drug over vehicle based on the following hypotheses:

H0:μactive−μvehicle=0

H1:μactive−μvehicle>0.

Where H0 is the null hypothesis, H1 the alternative hypotheses, μactive is the absolute change in the number of inflammatory lesions counts from Baseline to Day 84, and μvehicle is the absolute change in the number of inflammatory lesions counts from Baseline to Day 84.

Secondary and exploratory endpoints do not have a priori hypotheses but will be evaluated using appropriate statistical methods to inform statistical approaches for future studies.

### Analysis Datasets

This study will be evaluated using 3 analysis sets: intent-to-treat (ITT), per protocol (PP), and safety. Efficacy conclusions will be drawn from the ITT analysis set. The PP analysis set will be used to support the efficacy findings in the ITT analyses. Safety conclusions will be drawn from the safety analysis set.

The ITT analysis set includes all subjects who are randomized and is based on randomized study group, regardless of study drug received. The safety analysis set includes all subjects who are randomized, receive at least 1 confirmed dose of study drug, and have at least 1 post-Baseline assessment. The safety analysis set will be assessed based on study drug received, regardless of group to which subject was randomized. The PP analysis set includes all subjects in the ITT analysis set who complete the Day 84 visit without noteworthy study protocol violations, including compliance with study drug application, Day 84 visit window, and completion of efficacy evaluations on Day 84. The full definition of the PP population is given in the SAP which will be approved prior to database lock.

Subjects who have a documented lack of treatment effect or who are discontinued from the study due to an AE that was considered by the investigator to be study drug related will be included in the PP analysis set. Specific criteria for identifying the PP analysis set will be determined prior to breaking the blind.

Vehicle QD and Vehicle BID groups may be combined for analyses.

### Description of Statistical Methods

All statistical processing will be performed using SAS® 9.3 or higher. Demographics will be summarized by age, gender, race, ethnicity, height and weight. Summary statistics will be presented for change from Baseline in lesion counts (inflammatory and non-inflammatory separate and combined) and IGA. For continuous variables, the mean, standard deviation (SD), median, and range will be presented. Categorical variables will be summarized by frequency counts and percentages.

### Example 6

Residual cannabidiol concentrations for a range of compositions were measured before identifying the compositions most suitable for use in the dosage regimens of the present invention, as summarised in Table 4, below.

TABLE 4
Concentration of Cannabidiol (CBD) on skin after evaporation of volatile
solvents

| | | | | Final CBD |
|---|---|---|---|---|
| | | | | Concen- |
| | | | | tration |
| | Initial CBD | Volatile | Residual | After |
| | Concentration | Component(s) | solvent(s) | Evaporation |
| Formulation | % w/w | % w/w | % w/w | % w/w |
| 1 | 0.1 | 99.7 | 0.2 | 33.3 |
| 2 | 0.5 | 99.3 | 0.2 | 71.4 |
| 3 | 1.0 | 98.8 | 0.2 | 83.3 |
| 4 | 1.0 | 98.0 | 1.0 | 50.0 |
| 5 | 5.0 | 94.0 | 1.0 | 83.3 |
| 6 | 10.0 | 89.0 | 1.0 | 90.9 |
| 7 | 1.0 | 97.0 | 2.0 | 33.3 |
| 8 | 5.0 | 93.0 | 2.0 | 71.4 |
| 9 | 10.0 | 88.0 | 2.0 | 83.3 |
| 10 | 1.0 | 96.0 | 3.0 | 25.0 |
| 11 | 5.0 | 92.0 | 3.0 | 60.0 |
| 12 | 10.0 | 87.0 | 3.0 | 76.9 |

TABLE 5
Compositions for use in one or more of the abovementioned studies
Composition of BTX 1503 Topical Liquid and BTX 1503 G Topical Gel
Formulations

| | percent w/w | | | | |
|---|---|---|---|---|---|
| | BTX 1503 | BTX 1503 | BTX 1503 G | BTX 1503 G | |
| Ingredient | 2.5% CBD^{a} | 5% CBD^{a} | 2.5% CBD^{b} | 5% CBD^{b} | Function |
| Cannabidiol | 2.5 | 5.0 | 2.50 | 5.0 | Active ingredient |
| Dow Q7-9180 Silicone Fluid | 94.3 | 92.0 | 87.27 | 85.0 | Volatile solvent |
| 0.65 CST | | | | | |
| Dow Q7-9120 Silicone Fluid | 1.1 | 1.0 | 1.02 | 1.0 | Viscosity modifier |
| 12,500 CST | | | | | |
| Arlamol PS15E (PPG-15 | 2.1 | 2.0 | 1.02 | 1.0 | Emollient |
| Stearyl Ether) | | | | | |
| Dow 1515 Gum | — | — | 5.12 | 5.0 | Viscosity modifier |
| Isopropyl alcohol | — | — | 3.07 | 3.0 | Solvent |
| (anhydrous) | | | | | |
| Total | 100 | 100 | 100 | 100 | |

^{a}Formulation used for Clinical Study Nos. BTX.2017.001 (5% only) and BTX.2017.002, and BTX90DMPGLP (also known as Study No. 20111980).
^{b}Formulation to be used for Clinical Study No. BTX.2017.001.

Study BTX.2017.001 is presented above as Example 2, Study BTX.2017.002 is presented above as Example 3, and Study BTX.2018.001 is presented above as Example 4.

Numerous variations and modifications of the above-described modes of carrying out the various embodiments of this invention will be apparent to those skilled in the art, based on the above teachings related to the disclosed invention, without departing from the basic inventive concepts. The above embodiments of the invention are merely exemplary and should not be construed to be in any way limiting and all such variations and modifications are to be considered within the scope of the present invention, the nature of which is to be determined from the foregoing description.

1. A method for treating acne, comprising the administration of:
a) between 50 mg and 3000 mg of a topical liquid or gel composition comprising between 1% w/w and 15% w/w cannabinoid, wherein the cannabinoid is dissolved in the liquid or gel composition.

2. The method of claim 1, wherein the topical composition is administered to the skin between 1 and 5 times per day.

3. The method of claim 1, wherein the topical composition delivers between 20 mg and 400 mg of cannabinoid per administration.

4. The method of claim 1, wherein the total daily dose applied to the skin is between 20 mg and 2000 mg cannabinoid.

5. The method of claim 1 wherein:
a) the topical composition comprises 2.5% w/w or 5% w/w cannabinoid; and/or
b) the regime delivers 37.5 mg, 75 mg or 150 mg of cannabinoid per day.

6. The method of claim 1, wherein the cannabinoid is delivered in a composition comprising: (i) a volatile solvent; and (ii) a residual solvent that is less volatile than (i).

7. The method of claim 6, wherein the volatile solvent is a non-polymeric siloxane.

8. The method of claim 6, wherein the volatile solvent is a combination of a non-polymeric siloxane and a C₂-C₆ alcohol.

9. The method of claim 8, wherein the composition comprises 85-95% w/w siloxane and 1-10% wt/wt C₂-C₆ alcohol.

10. The method regime of claim 9, wherein the siloxane has two or three silicon atoms per molecule.

11. The method of claim 10 wherein the siloxane comprises hexamethyldisiloxane.

12. The method of claim 6, wherein the residual solvent is a compound from the list comprising: fatty acids, fatty acid alcohols, fatty alcohols, glycols, alkanes, ethers of any of these, and combinations thereof.

13. The method of claim 12, wherein the composition comprises 1-10% wt/wt of residual solvent.

14. The method of claim 13, wherein the residual solvent is a compound from the list comprising: alkyl polypropylene glycol, polyethylene glycol ether, oleyl alcohol, isostearyl alcohol, octyldodecyl alcohol, 2-hexyl decyl alcohol, isohexadecane.
